(19) United States
(12) Patent Application Publication
(10) Pub. No.: US 2026/0184842 A1
(43) Pub. Date: Jul. 2, 2026

(54) EPOXY RESIN CURING AGENT, INSULATING EPOXY RESIN, AND PREPARATION METHOD THEREOF

(71) Applicant: XI'AN JIAOTONG UNIVERSITY, Xi'an (CN)

(72) Inventors: Boya ZHANG, Xi'an (CN); Chenhao JIA, Xi'an (CN); Jie LI, Xi'an (CN); Xingwen LI, Xi'an (CN)

(73) Assignee: XI'AN JIAOTONG UNIVERSITY, Xi'an (CN)

(21) Appl. No.: 19/380,950

(22) Filed: Nov. 5, 2025

Related U.S. Application Data
(63) Continuation of application No. PCT/CN2025/078998, filed on Feb. 25, 2025.

(30) Foreign Application Priority Data
Dec. 30, 2024 (CN) 202411972784.4

(51) Int. Cl. C08G 59/50; C07C 211/36; C07C 211/50; C07C 211/52; C08G 59/24; C08J 3/21; C08K 3/22

(52) U.S. Cl. CPC C08G 59/504; C07C 211/36; C07C 211/50; C07C 211/52; C08G 59/245; C08G 59/5026; C08G 59/5033; C08J 3/212; C08K 3/22; C07C 2601/14; C08J 2363/02; C08K 2003/2227

(57) ABSTRACT

The present application belongs to a curing agent structure. An epoxy resin curing agent, an insulating epoxy resin, and a preparation method thereof are provided, including at least two benzene ring structures or at least two non-coplanar rigid alicyclic structures. Two adjacent benzene ring structures or non-coplanar rigid alicyclic structures are connected by methylene, and an amino functional group is arranged on each side of the integral structure. An ortho position of the amino functional group is provided with an electron-withdrawing substituent group or several sterically hindered substituent groups. By introducing benzene rings, the thermal stability of the obtained epoxy resin is enhanced. By introducing a high electron-withdrawing group and a large sterically hindered group or using a non-coplanar alicyclic structure with saturated electrons instead of a benzene ring, it can maintain good electrical properties in an environment with a high temperature gradient and a large electric field.

### CROSS-REFERENCE TO RELATED APPLICATIONS

The application claims priority to Chinese patent application No. 202411972784.4, filed on Dec. 30, 2024, the entire contents of which are incorporated herein by reference.

### TECHNICAL FIELD

The present application belongs to a curing agent structure, and specifically relates to an epoxy resin curing agent, an insulating epoxy resin, and a preparation method thereof.

### BACKGROUND

With the focus on carbon emissions and the rapid development of the renewable energy industry, large-scale energy transmission and utilization have become increasingly important, and the High Voltage Direct Current (HVDC) transmission technology is of great significance to the development of large-scale energy transmission and utilization. Currently, thermosetting polymers, particularly epoxy resin-based insulating materials, are widely used as electrical insulating materials in DC Gas-Insulated transmission Lines (GIL)/Gas Insulated Switchgear (GIS), bushings, Solid-State Transformers (SSTs), rectifiers, and other power equipment and electronic devices due to their excellent mechanical properties, corrosion resistance, and electrical properties.

However, as the voltage level and power density increase, the resistive consumption of a conductor and the heat dissipation inside an insulating material will cause the operating temperature of the insulating material to rise, which requires the insulating material to maintain excellent electrical properties at high temperatures. And under actions of the direct current, the electric field is resistively distributed, while the resistivity of epoxy resins is extremely sensitive to temperature changes. Under the action of high temperature and large electric fields, it is easy to cause electric field distortion and reduced insulation strength. The glass transition temperature of traditional anhydride-cured epoxy resins is only about 120° C. When the ambient operating temperature approaches this temperature, their electrical and mechanical properties will be greatly reduced. Therefore, it is of great significance to develop epoxy resin insulating materials that can still have excellent electrical properties at high temperatures for future energy networks.

At present, the main methods to improve the electrical performance of polymer materials at high temperatures include nano-doping technology and molecular design. Although nano-doping technology has great potential, it cannot ensure that nanoparticles are evenly dispersed inside the material. If the doping concentration is too high, it will lead to particle agglomeration problems, which will still hinder its practical application. In addition, the molecular design is further an effective method for optimizing high-performance polymers. However, this method is still in the experimental stage in designing epoxy resins with high thermal stability and high electrical properties, and the current methods mainly focus on directly improving the electrical insulation properties of polymers. Although the introduction of deep traps can increase the energy required to excite electrons at high temperatures, the dominant factor in the decline of epoxy resin performance is the molecular thermal motion caused by high temperatures. Therefore, it is urgent to find a way to improve the thermal stability and electrical performance of epoxy resin insulating materials.

### SUMMARY

### Technical Issues

The present application aims at the technical issues that nanoparticles are easy to agglomerate and disperse unevenly, and the electrical performance enhancement effect is unclear at high temperatures in the current method for improving the electrical properties of polymer materials at high temperatures. An epoxy resin curing agent, an insulating epoxy resin, and a preparation method thereof are then provided.

### Technical Solutions

To achieve the preceding purpose, the present application provides the following technical solutions:

In a first aspect, the present application proposes an epoxy resin curing agent, including:
at least two benzene ring structures or at least two non-coplanar rigid alicyclic structures;
two adjacent benzene ring structures or non-coplanar rigid alicyclic structures are connected by methylene to form an integral structure; and
an amino functional group is provided on each side of the integral structure, with an
electron-withdrawing substituent group or several sterically hindered substituent groups being disposed at an ortho position of the amino functional group.

Further, the electron-withdrawing substituent group is —Cl;

The sterically hindered substituent group is —CH₃ or —C₂H₅.

Further, the epoxy resin curing agent structure includes at least two non-coplanar rigid alicyclic structures;

The non-coplanar rigid alicyclic structure is C₆H₁₂.

Further, one or two sterically hindered substituent groups are present.

In a second aspect, the present application provides a preparation method of an insulating epoxy resin, including:
dispersing the preceding epoxy resin curing agent in diglycidyl ether bisphenol A (DGEBA) to obtain a first mixture;
adding alumina filler to the liquid phase mixture to obtain a second mixture;
heating the second mixture, degassing the second mixture under vacuum, and then casting it into a mold and deairing it under vacuum; and
obtaining an insulating epoxy resin, the finished product, after curing and cooling.

Further, the weight ratio of epoxy resin curing agent to diglycidyl ether bisphenol A (DGEBA) to alumina filler is (10-35):(15-45):(35-65).

Further, the curing conditions are as follows:
raising the temperature to 120-140° C. and holding for 60-120 min; raising the temperature to 160-180° C. and holding for 100-150 min; and finally raising the temperature to 180-220° C. and holding for 60-120 min.

Further, the method for dispersing the epoxy resin curing agent in diglycidyl ether bisphenol A (DGEBA) includes:
melting the epoxy resin curing agent at 100-120° C., and then stirring and dispersing the epoxy resin curing agent in diglycidyl ether bisphenol A (DGEBA) at 100-120° C.

Further, heat the second mixture so that the temperature condition for degassing the second mixture under vacuum is 100-140° C.

In a third aspect, the present application provides an insulating epoxy resin prepared by the preceding preparation method of an insulating epoxy resin.

### Beneficial Effects

Compared with the existing art, the present application has the following beneficial effects:

The present application proposes an epoxy resin curing agent, which introduces a diaryldiamine curing agent and utilizes the rigidity and planar structure of the benzene ring to improve the thermal stability of epoxy resins, increase the glass transition temperature of epoxy resins, and enable the epoxy resin to maintain its original performance at higher temperatures. The electrical properties of epoxy resins are improved by grafting different groups at the ortho position of the amino functional group. By grafting a high electron-withdrawing group at the ortho position of the amino functional group, its adsorption effect on electrons is used to reduce the π electron density on the benzene ring, thereby reducing the conductivity of the epoxy resin. Or grafting large sterically hindered groups such as methane and ethane at the ortho position of the amino functional group, use the sterically hindered effect and distorted conformation generated in the epoxy resin chain to block the charge transfer between different molecules caused by the benzene ring conjugation effect, thereby improving the electrical properties of epoxy resins. In addition, a non-planar rigid alicyclic structure with saturated electrons is used instead of benzene rings, and its weak conjugation effect is used to reduce the concentration of non-local electrons, thereby improving the electrical properties of epoxy resins. By modifying the epoxy resin curing agent using the preceding method, an insulating epoxy resin having both high thermal stability and high electrical properties can be prepared, thereby solving the problem of insulation failure in an environment with a high temperature gradient and a large electric field.

The present application further proposes a preparation method of an insulating epoxy resin. By introducing benzene rings, the thermal stability of the prepared epoxy resin is enhanced. By introducing a high electron-withdrawing group and a large sterically hindered group or using a non-coplanar alicyclic structure with saturated electrons instead of a benzene ring, the volume resistivity of the epoxy resin at room temperature and high temperature is significantly improved, and it can maintain good electrical properties in an environment with a high temperature gradient and a large electric field.

The present application further provides an insulating epoxy resin prepared by the preceding preparation method of an insulating epoxy resin, which has all the advantages of the preceding epoxy resin curing agent and the preparation method of the insulating epoxy resin.

### BRIEF DESCRIPTION OF DRAWINGS

To clearly illustrate the technical solution of embodiments of the present application, the drawings required for describing the embodiments will be briefly introduced below. It should be understood that the following drawings only show some embodiments of the present application and should not be regarded as limiting the scope. For those of ordinary skill in the art, other related drawings can be obtained further from these drawings without creative work.

FIG. 1 is a Fourier transform infrared (FTIR) spectrum of diglycidyl ether bisphenol A (DGEBA), insulating epoxy resins obtained in Embodiments 1-6 of the present application, and the Comparative Embodiment.

FIG. 2 is a glass transition temperature diagram of insulating epoxy resins obtained in Embodiments 1-6 of the present application and the Comparative Embodiment.

FIG. 3 is a DC breakdown strength and shape parameter diagram of insulating epoxy resins obtained in Embodiments 1-6 of the present application and the Comparative Embodiment at room temperature.

FIG. 4 is a graph showing the volume resistivity results of insulating epoxy resins obtained in Embodiments 1-6 of the present application and the Comparative Embodiment at 80° C.

### DETAILED DESCRIPTION OF THE EMBODIMENTS

In order to make the purpose, technical solution, and advantages of the embodiments of the present application clearer, the following will combine the drawings in the embodiments of the present application to clearly and completely describe the technical solutions in the embodiments of the present application. Obviously, the described embodiments are some but not all embodiments of the present application. The components of the embodiments of the present application generally described and shown in the drawings herein can be arranged and designed in various different configurations.

Therefore, the following detailed description of the embodiments of the present application provided in the accompanying drawings is not intended to limit the scope of the claimed application, but only represents selected embodiments of the present application. Based on the embodiments described herein, all other embodiments obtained by those of ordinary skill in the art without creative work are within the scope of protection of the present application.

It should be noted that similar reference numerals and letters represent similar items in the following figures, so once an item is defined in one figure, it does not need to be further defined and explained in subsequent figures.

In the description of the embodiments of the present application, it should be noted that if the terms “upper”, “lower”, “horizontal”, “inside”, and so on indicate an orientation or positional relationship based on the orientation or positional relationship shown in the accompanying drawings, or the orientation or positional relationship in which the invention product is usually placed when used, they are only for the convenience of describing the present application and simplifying the description, and do not indicate or imply that the device or component referred to must have a specific orientation. Constructed and operated in a specific orientation, it cannot be understood as a limitation of the present application. In addition, the terms “first”, “second”, and so on are only used to distinguish descriptions and cannot be understood as indicating or implying relative importance.

In addition, when the term “horizontal” appears, it does not mean that the component is required to be absolutely horizontal, but can be slightly tilted. For example, “horizontal” only means that its direction is more horizontal than “vertical”, which does not mean that the structure must be completely horizontal, but can be slightly tilted.

In the description of the embodiments of the present application, it should be noted further that unless otherwise clearly specified and limited, the terms “setting”, “installation”, “be connected with” and “be connected to” should be understood in a broad sense. For example, they can be fixed connections, detachable connections, or integrated connections. It can be a mechanical connection or an electrical connection. It can be directly connected or indirectly connected through an intermediate medium, and it can be the internal connection of two components. For those of ordinary skill in the art, the specific meaning of the preceding terms in the present application can be understood according to the specific situation.

As the voltage level and power density keep increasing, the problems of resistive consumption of a conductor and the heat dissipation inside an insulating material have become increasingly prominent, resulting in a gradual increase in the operating temperature of the insulating material. This requires that the insulating material must still maintain excellent electrical performance in high temperature environments. However, under the action of a DC electric field, the electric field is resistively distributed, and the resistivity of epoxy resins is extremely sensitive to temperature changes. High temperature and large electric field environments are prone to cause electric field distortion and reduced insulation strength. The glass transition temperature of traditional anhydride-cured epoxy resins is only about 120° C. When the ambient operating temperature approaches this temperature, their electrical and mechanical properties will be greatly reduced.

Based on the preceding situation, the present application proposes an epoxy resin curing agent, an insulating epoxy resin and a preparation method thereof. The present application is described in detail below with reference to embodiments and drawings.

First, the present application proposes an epoxy resin curing agent, including two structures:
(1) At least two benzene ring structures, among them, two adjacent benzene ring structures are connected by methylene to form an integral structure, and methylene is arranged at the 1,4 substituent position of the benzene ring structure. An amino functional group is provided on each side of the integral structure, with an electron-withdrawing substituent group or several sterically hindered substituent groups being disposed at an ortho position of the amino functional group.

The connection between two adjacent benzene ring structures by methylene makes the entire molecule have a certain flexibility while maintaining the rigidity of the benzene ring. An amino functional group is arranged on each side of the integral structure as a key part for subsequent curing reaction with epoxy resins. An ortho position of the amino functional group is provided with an electron-withdrawing substituent group or several sterically hindered substituent groups, which can affect the electron cloud density and sterically hindered of the amino functional group, thereby affecting its reactivity with epoxy resins and the performance of the cured product. For the integral structure, due to the presence of the benzene ring structure, this curing agent can have good heat resistance and chemical resistance. With introduction of high electron-withdrawing groups, their strong adsorption effect on electrons can be used to reduce the electron cloud density on the benzene ring, thereby increasing the resistivity of the material. With introduction of sterically hindered groups, the charge transfer between different molecules can be hindered to increase the resistivity of the final product.
(2) At least two non-coplanar rigid alicyclic structures, among them, two adjacent non-coplanar rigid alicyclic structures are connected by methylene to form an integral structure, one amino functional group is arranged on each side of the integral structure, and an electron-withdrawing substituent group or several sterically hindered substituent groups are arranged at an ortho position of the amino functional group.

In the second structure, a non-planar structure instead of a benzene ring is used, which reduces the local electron concentration through a weak conjugation effect of the non-planar structure, thereby increasing the resistivity of the final product.

Specifically, some embodiments of the preceding epoxy resin curing agent may:

1. Include two benzene ring structures:
(1) The electron-withdrawing substituent group is a high electron-withdrawing group. High electron-withdrawing groups refer to groups with strong electron attraction ability, which can significantly reduce the density of electron clouds on benzene rings. They usually have strong electronegativity and can attract electrons through induction effect and conjugation effect. Common high electron-withdrawing groups can be nitro, tertiary amine cations, halogen atoms, and so on.

For example, the halogen atom is adopted:

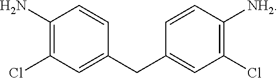 [SMILES: Nc1ccc(Cc2ccc(N)c(Cl)c2)cc1Cl]

(2) The sterically hindered substituent group may be a large sterically hindered substituent group. Large sterically hindered substituent groups refer to substituent groups with large volume in the molecule. Due to its large volume, it occupies a certain range in space, causing the molecule as a whole to show a certain distortion or deformation, which will affect the spatial orientation, three-dimensional structure, and chemical properties of the molecule.

For example, —CH₃ is adopted:

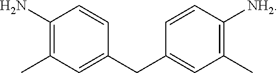

Two-CH₃s are adopted:

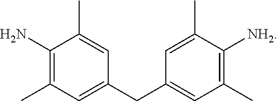

—C₂H₅ is adopted:

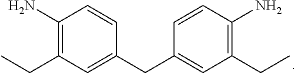 [SMILES: CCc1cc(Cc2ccc(N)c(CC)c2)ccc1N]

Two —C₂H₅s are adopted:

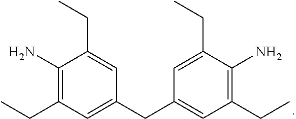 [SMILES: CCc1cc(Cc2cc(CC)c(N)c(CC)c2)cc(CC)c1N]

2. Include two non-coplanar rigid alicyclic structures:

The non-coplanar rigid alicyclic structure is C₆H₁₂:

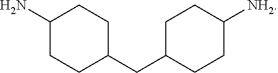 [SMILES: NC1CCC(CC2CCC(N)CC2)CC1]

The preceding curing agent structure, by introducing a benzene ring, enhances the thermal stability of the obtained epoxy resin. By introducing a high electron-withdrawing group and a large sterically hindered group or using a non-coplanar alicyclic structure with saturated electrons instead of a benzene ring, the volume resistivity of the epoxy resin at room temperature and high temperature is significantly improved, and it can maintain good electrical properties in an environment with a high temperature gradient and a large electric field.

The present application further provides a preparation method of an insulating epoxy resin using the preceding curing agent, which may include:
S1, dispersing the epoxy resin curing agent of the present application in diglycidyl ether bisphenol A (DGEBA) to obtain a first mixture.
S2, adding alumina filler to the liquid phase mixture to obtain a second mixture.

Alumina fillers can increase the mechanical strength, heat resistance, and thermal conductivity of epoxy resins. Moreover, alumina further has good insulation properties and high temperature stability.
S3, heating the second mixture, degassing the second mixture under vacuum, and then casting it into a mold and deairing it under vacuum.

Degassing under vacuum conditions can remove bubbles from the mixture and improve the compactness and insulation properties of the finished product. Pour the degassed mixture into a pre-prepared mold and degas it under vacuum conditions to further remove bubbles and ensure that the mixture is evenly distributed in the mold.
S4, obtaining an insulating epoxy resin, the finished product, after curing and cooling.

The temperature and time can further be controlled during the curing process to ensure that the epoxy resin is fully cured. After curing is completed, the pre-finished product can be taken out of the mold and cooled. After cooling, an insulating epoxy resin, the finished product, can be obtained.

The following are some embodiments:

### Embodiment 1

Take 4,4′-methylenebis-o-chloroaniline (MOCA) with two benzene ring structures and a strong electron-withdrawing group —Cl as an epoxy resin curing agent, namely:

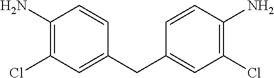 [SMILES: Nc1ccc(Cc2ccc(N)c(Cl)c2)cc1Cl]

The preparation method of an insulating epoxy resin is as follows:
(1) Preheat diglycidyl ether bisphenol A (DGEBA) at 60° C. for 8 h in advance. Melt 4,4′-methylenebis-o-chloroaniline (MOCA) at 100° C., stir and disperse MOCA in diglycidyl ether bisphenol A (DGEBA) at a high speed for 30 min at the same temperature to obtain a first mixture.
(2) Add alumina fillers to the first mixture of epoxy resin curing agent and diglycidyl ether bisphenol A (DGEBA) to obtain a second mixture. Heat and stir the second mixture, and degas the second mixture under vacuum at 120° C. for 30 min. Spray the mold with a release agent and preheat it to 120° C. Subsequently, cast the second mixture into the preheated mold and then degas it under vacuum at the same temperature for 10 min. Specifically, the second mixture contains 10 parts of 4,4′-methylenebis-o-chloroaniline (MOCA), 15 parts of diglycidyl ether bisphenol A (DGEBA), and 35 parts of Al₂O₃ micron base filler.
(3) Carry out the cross-linking reaction in a stepwise heating manner, and raise the curing environment temperature to 120° C. and hold for 60 min, raise the temperature to 160° C. and hold for 120 min, and finally raise the temperature to 180° C. and hold for 120 min. After natural cooling to room temperature, take an insulating epoxy resin material of about 0.3 mm thick out from the mold and name it EP-MOCA.

### Embodiment 2

Take 4,4′-methylenebis-o-toluidine (MBOT) with two benzene ring structures and a large sterically hindered group —CH₃ as an epoxy resin curing agent, namely:

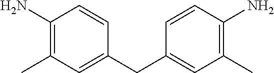

The preparation method of an insulating epoxy resin is as follows:
(1) Preheat diglycidyl ether bisphenol A (DGEBA) at 80° C. for 6 h in advance. Melt 4,4′-methylenebis-o-toluidine (MBOT) at 110° C., and stir and disperse MBOT in diglycidyl ether bisphenol A (DGEBA) at a high speed for 35 min at the same temperature to obtain a first mixture.
(2) Add alumina fillers to the first mixture of epoxy resin curing agent and diglycidyl ether bisphenol A (DGEBA) to obtain a second mixture. Heat and stir the second mixture, and degas the second mixture under vacuum at 100° C. for 50 min. Spray the mold with a release agent and preheat it to 100° C. Subsequently, cast the second mixture into the preheated mold and then degas it under vacuum at the same temperature for 20 min. Specifically, the second mixture contains 35 parts of 4,4′-methylenebis-o-toluidine (MBOT), 45 parts of diglycidyl ether bisphenol A (DGEBA), and 65 parts of Al₂O₃ micron base filler.
(3) Carry out the cross-linking reaction in a stepwise heating manner, and raise the curing environment temperature to 130° C. and hold for 80 min, raise the temperature to 170° C. and hold for 100 min, and finally raise the temperature to 200° C. and hold for 100 min. After natural cooling to room temperature, take an insulating epoxy resin material of about 0.3 mm thick out from the mold and name it EP-MBOT.

### Embodiment 3

Take 4,4-methylenebis(2,6-dimethylaniline) (MBMDA) with two benzene ring structures and a large sterically hindered group —CH₃ as an epoxy resin curing agent, namely:

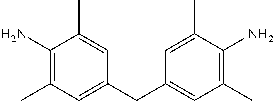

The preparation method of an insulating epoxy resin is as follows:
(1) Preheat diglycidyl ether bisphenol A (DGEBA) at 75° C. for 6 h in advance. Melt 4,4′-methylenebis(2,6-dimethylaniline) (MBMDA) at 105° C., and stir and disperse MBMDA in diglycidyl ether bisphenol A (DGEBA) at a high speed for 42 min at the same temperature to obtain a first mixture.
(2) Add alumina fillers to the first mixture of epoxy resin curing agent and diglycidyl ether bisphenol A (DGEBA) to obtain a second mixture. Heat and stir the second mixture, and degas the second mixture under vacuum at 140° C. for 45 min. Spray the mold with a release agent and preheat it to 140° C. Subsequently, cast the second mixture into the preheated mold and then degas it under vacuum at the same temperature for 15 min. Specifically, the second mixture contains 20 parts of 4,4-methylenebis(2,6-dimethylaniline) (MBMDA), 30 parts of diglycidyl ether bisphenol A (DGEBA), and 50 parts of Al₂O₃ micron base filler.
(3) Carry out the cross-linking reaction in a stepwise heating manner, and raise the curing environment temperature to 120° C. and hold for 70 min, raise the temperature to 180° C. and hold for 120 min, and finally raise the temperature to 220° C. and hold for 60 min. After natural cooling to room temperature, take an insulating epoxy resin material of about 0.3 mm thick out from the mold and name it EP-MBMDA.

### Embodiment 4

Take 4,4′-methylenebis-o-ethylaniline (MOEA) with two benzene ring structures and a large sterically hindered group —C₂H₅ as an epoxy resin curing agent, namely:

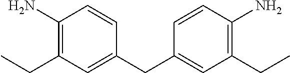 [SMILES: CCc1cc(Cc2ccc(N)c(CC)c2)ccc1N]

The preparation method of an insulating epoxy resin is as follows:
(1) Preheat diglycidyl ether bisphenol A (DGEBA) at 80° C. for 7 h in advance. Melt 4,4′-methylenebis-o-ethylaniline (MOEA) at 115° C., and stir and disperse MOEA in diglycidyl ether bisphenol A (DGEBA) at a high speed for 40 min at the same temperature to obtain a first mixture.
(2) Add alumina fillers to the first mixture of epoxy resin curing agent and diglycidyl ether bisphenol A (DGEBA) to obtain a second mixture. Heat and stir the second mixture, and degas the second mixture under vacuum at 130° C. for 50 min. Spray the mold with a release agent and preheat it to 130° C. Subsequently, cast the second mixture into the preheated mold and then degas it under vacuum at the same temperature for 25 min. Specifically, the second mixture contains 20 parts of 4,4′-methylenebis-o-ethylaniline (MOEA), 30 parts of diglycidyl ether bisphenol A (DGEBA), and 50 parts of Al₂O₃ micron base filler.
(3) Carry out the cross-linking reaction in a stepwise heating manner, and raise the curing environment temperature to 140° C. and hold for 90 min, raise the temperature to 170° C. and hold for 100 min, and finally raise the temperature to 200° C. and hold for 90 min. After natural cooling to room temperature, take an insulating epoxy resin material of about 0.3 mm thick out from the mold and name it EP-MOEA.

### Embodiment 5

Take 4,4′-methylene-(bis(2,6-diethylaniline) (MDEA) with two benzene ring structures and a large sterically hindered group —C₂H₅ as an epoxy resin curing agent, namely:

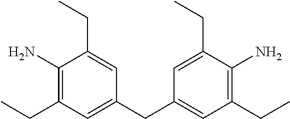 [SMILES: CCc1cc(Cc2cc(CC)c(N)c(CC)c2)cc(CC)c1N]

The preparation method of an insulating epoxy resin is as follows:
(1) Preheat diglycidyl ether bisphenol A (DGEBA) at 80° C. for 8 h in advance. Melt 4,4′-methylene-(bis(2,6-diethylaniline) (MDEA) at 120° C., and stir and disperse MDEA in diglycidyl ether bisphenol A (DGEBA) at a high speed for 50 min at the same temperature to obtain a first mixture.
(2) Add alumina fillers to the first mixture of epoxy resin curing agent and diglycidyl ether bisphenol A (DGEBA) to obtain a second mixture. Heat and stir the second mixture, and degas the second mixture under vacuum at 140° C. for 50 min. Spray the mold with a release agent and preheat it to 140° C. Subsequently, cast the second mixture into the preheated mold and then degas it under vacuum at the same temperature for 30 min. Specifically, the second mixture contains 20 parts of 4,4′-methylene-(bis(2,6-diethylaniline) (MDEA), 30 parts of diglycidyl ether bisphenol A (DGEBA), and 50 parts of Al₂O₃ micron base filler.
(3) Carry out the cross-linking reaction in a stepwise heating manner, and raise the curing environment temperature to 140° C. and hold for 120 min, raise the temperature to 180° C. and hold for 150 min, and finally raise the temperature to 220° C. and hold for 120 min. After natural cooling to room temperature, take an insulating epoxy resin material of about 0.3 mm thick out from the mold and name it EP-MDEA.

### Embodiment 6

Take para-diaminodicyclohexylmethane (PACM) containing a non-coplanar saturated alicyclic structure C₆H₁₂ as an epoxy resin curing agent, namely:

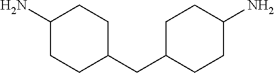 [SMILES: NC1CCC(CC2CCC(N)CC2)CC1]

The preparation method of an insulating epoxy resin is as follows:
(1) Preheat diglycidyl ether bisphenol A (DGEBA) at 60° C. for 6 h in advance. Melt para-diaminodicyclohexylmethane (PACM) at 100° C., and stir and disperse PACM in diglycidyl ether bisphenol A (DGEBA) at a high speed for 30 min at the same temperature to obtain a first mixture.
(2) Add alumina fillers to the first mixture of epoxy resin curing agent and diglycidyl ether bisphenol A (DGEBA) to obtain a second mixture. Heat and stir the second mixture, and degas the second mixture under vacuum at 100° C. for 30 min. Spray the mold with a release agent and preheat it to 100° C. Subsequently, cast the second mixture into the preheated mold and then degas it under vacuum at the same temperature for 10 min. Specifically, the second mixture contains 20 parts of para-diaminodicyclohexylmethane (PACM), 30 parts of diglycidyl ether bisphenol A (DGEBA), and 50 parts of Al₂O₃ micron base filler.
(3) Carry out the cross-linking reaction in a stepwise heating manner, and raise the curing environment temperature to 120° C. and hold for 60 min, raise the temperature to 160° C. and hold for 100 min, and finally raise the temperature to 180° C. and hold for 60 min. After natural cooling to room temperature, take an insulating epoxy resin material of about 0.3 mm thick out from the mold and name it EP-PACM.

### COMPARATIVE EMBODIMENT

Take 4,4′-diaminodiphenylmethane (DDM) as an epoxy resin curing agent, the preparation method of an insulating epoxy resin is as follows:
(1) Preheat diglycidyl ether bisphenol A (DGEBA) at 70° C. for 7 h in advance. Melt 4,4′-diaminodiphenylmethane (DDM) at 110° C., and stir and disperse DDM in diglycidyl ether bisphenol A (DGEBA) at a high speed for 40 min at the same temperature to obtain a first mixture.
(2) Add alumina fillers to the liquid phase mixture of epoxy resin curing agent and diglycidyl ether bisphenol A (DGEBA) to obtain a second mixture. Heat and stir the second mixture, and degas the second mixture under vacuum at 120° C. for 40 min. Spray the mold with a release agent and preheat it to 120° C. Subsequently, cast the second mixture into the preheated mold and then degas it under vacuum at the same temperature for 20 min. Specifically, the second mixture contains 20 parts of 4,4′-diaminodiphenylmethane (DDM), 30 parts of epoxy resin E51, and 50 parts of Al₂O₃ micron base filler.
(3) Carry out the cross-linking reaction in a stepwise heating manner, and raise the curing environment temperature to 130° C. and hold for 90 min, raise the temperature to 170° C. and hold for 125 min, and finally raise the temperature to 210° C. and hold for 80 min. After natural cooling to room temperature, take an insulating epoxy resin material of about 0.3 mm thick out from the mold and name it EP-DDM.

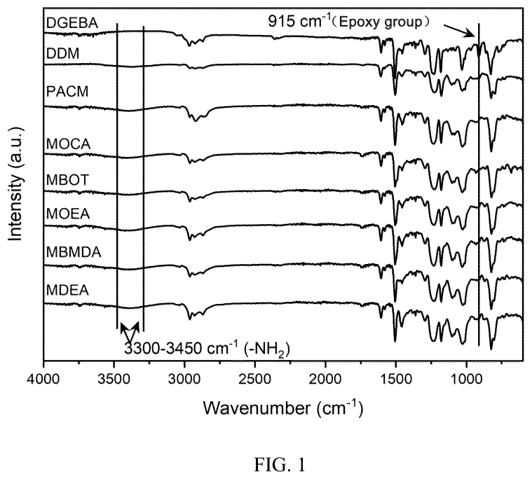

FIG. 1 is a Fourier transform infrared (FTIR) spectrum of diglycidyl ether bisphenol A (DGEBA), insulating epoxy resins obtained in Embodiments 1-6 of the present application, and the Comparative Embodiment. As can be seen from FIG. 1, the absorption peak at 915 cm⁻¹ disappears, and the characteristic peak related to the epoxy group disappears, indicating that the addition reaction between the amine and the epoxy ring has been completed. The disappearance of the N—H stretching vibration absorption peak in the range of 3,300-3,450 cm⁻¹ further confirms the consumption of —NH₂ functional groups in the curing agent.

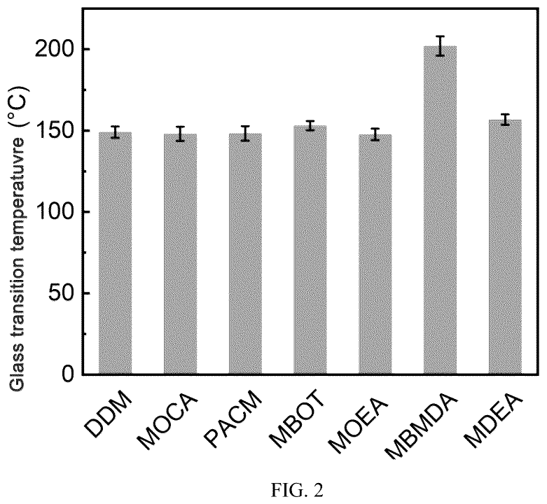

FIG. 2 is a glass transition temperature diagram of insulating epoxy resins obtained in Embodiments 1-6 of the present application and the Comparative Embodiment. For the insulating epoxy resins prepared in Embodiments 1-6 and the Comparative Embodiment, their glass transition temperatures are much higher than those of traditional anhydride-cured epoxy resins (about 120° C.), and the glass transition temperature of EP-MBMDA reaches 200° C. It can be seen that the introduction of benzene rings leads to an increase in the glass transition temperature of the resin, and since cyclohexane further has a certain degree of rigidity, the glass transition temperature of EP-PACM is correspondingly higher.

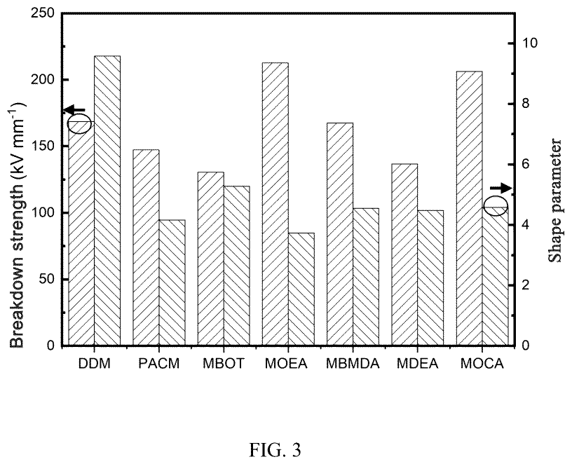

FIG. 3 is a DC breakdown strength and shape parameter diagram of insulating epoxy resins obtained in Embodiments 1-6 of the present application and the Comparative Embodiment at room temperature. Among them, the breakdown field strength is the Weibull breakdown strength. It can be seen that epoxy resin curing agents containing strong electron-withdrawing groups and large sterically hindered groups all show high electrical properties at room temperature. And for EP-MOEA and EP-MOCA, their breakdown strength exceeds 200 kV/mm, which is higher than the performance of traditional anhydride-cured epoxy resins.

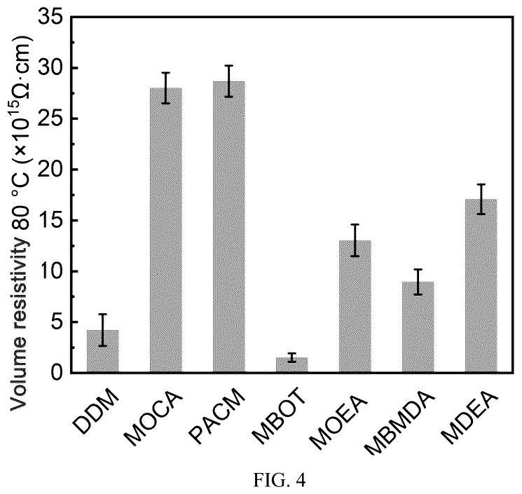

FIG. 4 is a graph showing the volume resistivity results of insulating epoxy resins obtained in Embodiments 1-6 of the present application and the Comparative Embodiment at 80° C. It can be found that the volume resistivity of EP-MOCA and EP-PACM at 80° C. exceeds 2.5×10¹⁵ Ω·cm, which is much higher than the performance of traditional anhydride-cured epoxy resins.

Except for EP-MBOT, the volume resistivity of insulating epoxy resins prepared in other embodiments at 80° C. is higher than that of EP-DDM, indicating that epoxy resin curing agents containing strong electron-withdrawing groups and large sterically hindered groups, and using non-coplanar saturated alicyclic structures instead of benzene rings can enable insulating epoxy resins to maintain high volume resistivity at high temperatures.

For current polymer electrolyte materials, their electrical properties and thermal stability are often mutually exclusive. An increase in temperatures often leads to the degradation of insulation performance. In order to develop epoxy resins with both high thermal stability and high electrical properties, the present application provides a modified epoxy resin curing agent, a preparation method of an insulating epoxy resin, and the insulating epoxy resin prepared by the preparation method. The thermal stability of epoxy resins is enhanced by introducing benzene rings. By grafting high electron-withdrawing groups or large sterically hindered groups at an ortho position of the amino functional group on the benzene ring, the π electrons concentration on the benzene ring can be reduced or the charge transfer between different molecules caused by the conjugation effect of the benzene ring can be blocked, thereby improving the electrical properties of epoxy resins. In addition, according to the present application, a non-planar rigid alicyclic structure with saturated electrons is used instead of benzene rings, and its weak conjugation effect is used to reduce the concentration of non-local electrons, thereby improving the electrical properties of epoxy resins. The epoxy resin curing agent obtained by the preceding method is used to prepare a high-resistance insulating epoxy resin material. The volume resistivity of the obtained insulating epoxy resin material at room temperature and high temperature has been significantly improved, which is of great significance for solving the insulation failure problem in an environment with a high temperature gradient and a large electric field.

The preceding are only preferred embodiments of the present application and are not intended to limit the present application. For those skilled in the art, the present application may have various changes and variations. Any modifications, equivalent substitutions, improvements, and so on made within the spirit and principles of the present application shall be included in the scope of protection of the present application.

What is claimed is:

1. An epoxy resin curing agent, comprising:
at least two benzene ring structures or at least two non-coplanar rigid alicyclic structures;
two adjacent benzene ring structures or non-coplanar rigid alicyclic structures are connected by methylene to form an integral structure; and
an amino functional group is provided on each side of the integral structure, with an electron-withdrawing substituent group or several sterically hindered substituent groups being disposed at an ortho position of the amino functional group.

2. The epoxy resin curing agent according to claim 1, wherein the electron-withdrawing substituent group is —Cl; and
the sterically hindered substituent group is —CH₃ or —C₂H₅.

3. The epoxy resin curing agent according to claim 1, wherein the epoxy resin curing agent structure comprises at least two non-coplanar rigid alicyclic structures; and
the non-coplanar rigid alicyclic structure is C₆H₁₂.

4. The epoxy resin curing agent according to claim 1, wherein one or two sterically hindered substituent groups are present.

5. A preparation method of an insulating epoxy resin, comprising the following steps:
dispersing the epoxy resin curing agent according to claim 1 in diglycidyl ether bisphenol A (DGEBA) to obtain a first mixture;
adding alumina filler to the liquid phase mixture to obtain a second mixture;
heating the second mixture, degassing the second mixture under vacuum, and then casting it into a mold and deairing it under vacuum; and
obtaining an insulating epoxy resin, the finished product, after curing and cooling.

6. The preparation method of an insulating epoxy resin according to claim 5, wherein the weight ratio of epoxy resin curing agent to diglycidyl ether bisphenol A (DGEBA) to alumina filler is (10-35):(15-45):(35-65).

7. The preparation method of an insulating epoxy resin according to claim 5, wherein the curing conditions are as follows:
raising the temperature to 120-140° C. and holding for 60-120 min; raising the temperature to 160-180° C. and holding for 100-150 min; and finally raising the temperature to 180-220° C. and holding for 60-120 min.

8. The preparation method of an insulating epoxy resin according to claim 5, wherein the method for dispersing the epoxy resin curing agent in diglycidyl ether bisphenol A (DGEBA) comprises:
melting the epoxy resin curing agent at 100-120° C., and then stirring and dispersing the epoxy resin curing agent in diglycidyl ether bisphenol A (DGEBA) at 100-120° C.

9. The preparation method of an insulating epoxy resin according to claim 5, wherein the second mixture is heated, and the temperature condition for degassing of the second mixture under vacuum is 100-140° C.

10. An insulating epoxy resin, being prepared by the preparation method of an insulating epoxy resin according to claim 5.
